(19) United States
(12) Patent Application Publication
(10) Pub. No.: US 2006/0275895 A1
(43) Pub. Date: Dec. 7, 2006

(54) Biogas producing facility with anaerobic hydrolysis

(75) Inventors: Jan Jensen, Roskilde (DK); Preben Jensen, Odder (DK)

Correspondence Address: EDWARDS & ANGELL, LLP, P.O. BOX 55874, BOSTON, MA, 02205, US

(73) Assignee: Bio-Circuit, Hinnerup (DK)

(21) Appl. No.: 10/561,875

(22) PCT Filed: Jun. 28, 2004
(86) PCT No.: PCT/DK04/00462

§ 371 Date: May 5, 2006

(30) Foreign Application Priority Data
Jun. 27, 2003 (DK) PA 2003 00978
Aug. 14, 2003 (DK) PA 2003 01166

(51) Int. Cl. C12M 1/107; C12P 5/02

(52) U.S. Cl. 435/300.1; 435/167; 435/294.1; 210/603; 48/127.7

(57) ABSTRACT

The present invention on relates to a method and a system for conversion of organic waste into biogas, i.e. a methane containing gas, with an improved efficiency and economy. The system comprises a reactor (3) for holding organic waste for production of biogas by digestion and having an output for digested waste, and an anaerobic tank (6) that is connected to the reactor (3) output for anaerobic hydrolysis of the digested waste and having an output for hydrolysed material that is connected to an input of the reactor for adding hydrolysed material to the content of the reactor. The anaerobic hydrolysis process makes the energy content of material that has not been digested in the reactor available for bacterial digestion and thus, the hydrolysed material is fed back into the reactor for further bacterial conversion into biogas.

### FIELD OF THE INVENTION

The present invention relates to a method and a system for conversion of organic waste into biogas, i.e. a methane containing gas, with an improved efficiency and economy.

### BACKGROUND OF THE INVENTION

Typically, today's biogas producing facilities depend on supply of industrial waste containing fat to be economically feasible. Fat has a high energy to weight ratio, which makes it a useful input for biogas producing facilities. There is a high demand for industrial waste for this purpose, which has made it a rather expensive and limited resource.

Thus, there is a need for a biogas producing facility that makes it possible to substitute industrial waste with other materials, e.g. other waste materials.

### SUMMARY OF THE INVENTION

According to the present invention, the above and other objects are fulfilled by a biogas producing facility comprising a first reactor for holding organic waste for production of biogas by digestion and having an output for digested waste, and an anaerobic tank that is connected to the reactor output for anaerobic hydrolysis of the digested waste, and having an output for hydrolysed material that is connected to an input of a second reactor for adding hydrolysed material to the content of the reactor.

In one embodiment of the invention, the first reactor also constitutes the second reactor.

The anaerobic hydrolysis process makes the energy content of material that has not been digested in the reactor available for bacterial digestion and thus, the hydrolysed material is fed into a second reactor, or, is fed back into the first reactor for further bacterial conversion into biogas.

The anaerobic hydrolysis process significantly increases the produced amount of biogas compared to a similar facility without the hydrolysis process.

Provision of anaerobic hydrolysis after digestion in the first reactor has the advantage that the amount of material to be processed in the anaerobic hydrolysis tank is kept at a minimum since the digestible part of the material has already been digested in the reactor. This reduces the required capacity of the anaerobic tank and related interconnecting systems thereby reducing investments and operational cost.

Further, anaerobic hydrolysis after digestion provides more energy than hydrolysis before digestion. This is believed to be caused by the fact that doing a hydrolysis process on a biomass with a high content of volatile and easily digestible and reactive volatiles induces a tendency for constituents of organic matter to denature or condense during hydrolysis into derivatives of organic matter that cannot be digested in the reactor. Therefore such materials may advantageously be digested in a reactor before hydrolysis.

Preferably, the anaerobic hydrolysis in the anaerobic tank is performed at a pressure that is substantially equal to or higher than the saturation vapour pressure.

It is a further advantage of the present invention that no further chemicals are added to assist the anaerobic hydrolysis process.

Surprisingly, it has been found that the output of the anaerobic hydrolysis substantially does not smell.

The hydrolysis process operates effectively on various materials, such as planting stock, such as straw, fibres, and similar fibre containing materials etc, sludge, such as biological sludge from sewage treatment plants, etc, bacterial material, animal feed remains, animal remains, etc.

Preferably, the reactor is an anaerobic reactor due to its low operational cost.

In a preferred embodiment of the invention, the biogas producing facility further comprises a separator that is connected to the first reactor output for selective separation of particles larger than a predetermined threshold size from the digested waste and having an output for the separated particles that is connected to the anaerobic tank for hydrolysis of the separated large particles.

Larger particles constitute most of the biological substance and thus, the useful biological substance is separated from the material that has been digested in the first reactor for further processing in the hydrolysis tank. This further reduces the required capacity of the anaerobic tank and related interconnecting systems, which in turn further reduces investments and cost.

The smaller particles have a large content of biological dry matter that can not be digested, for example lignin-like substances, salts of phosphor, etc, which it is not desirable to feed into the hydrolysis tank. Thus, the dry matter subjected to subsequent hydrolysis has low phosphor content.

In accordance with the present invention, the separation efficiency may be enhanced by adding precipitation agents or polymers whereby the particle size upstream the separation unit is increased leading to improved retention of solids for downstream hydrolysis.

For hydrolysis of sludge from wastewater treatment plants, the threshold size is preferably 1.0 mm, and more preferred 2.0 mm.

For hydrolysis of straw or similar material, the threshold size is preferably 0.2 cm, more preferred 0.5 cm, even more preferred 1.0 cm, still more preferred 1.5 cm, and most preferred 2.0 cm.

The separator may further comprise a dewatering device for dewatering of the separated particles.

The amount of substance entering into the hydrolysis tank is preferably less than 50% of the total amount of substance provided to the facility.

Hydrolysis is preferably performed at a pressure that is substantially equal to or higher than the saturation vapour pressure.

The pressure may be substantially equal to the ambient pressure, i.e. approximately 1 atmosphere, for provision of a simple and inexpensive hydrolysis system.

For some materials, performing the hydrolysis at higher pressures than ambient pressure, such as the saturation pressure at a temperature of 125° C., 190° C., etc may optimise the efficiency and economics of the biogas producing facility. Increased temperature decreases the duration of the hydrolysis. For example, hydrolysis may be performed at a temperature in the range from 50° C.-75° C. for 0,25 to 24 hours, or at a temperature in the range from 70° C.-100° C. for 0,25 to 16 hours, such as for 4 to 10 hours, or at a temperature in the range from 100° C.-125° C. for 0.25 to 8 hours, such as for 3 to 6 hours, or at a temperature in the range from 125° C.-150° C. for 0.25 to 6 hours, such as for 2 to 4 hours, or at a temperature in the range from 150° C.-175° C. for 0.25 to 4 hours, such as for 1 to 2 hours, or at a temperature in the range from 175° C.-200° C. for 0.25 to 2 hours, such as for 0.25 to 1 hours.

The biogas producing facility may further comprise a partitioning device for partitioning of organic waste and having an output for supplying the partitioned waste to the reactor.

The biogas producing facility according to the present invention has made it possible to substitute industrial waste with organic waste, such as corn, grass, dry grass, straw, silage, animal remains, etc. The straw may for example be fresh or dry straw or straw contained in livestock dung or in deep bedding. Thus, in a preferred embodiment, livestock dung mixed with straw is fed into the reactor. Straw has a dry matter content of 90-95% and in spite of the fact that the fat content of straw is very low; it has a significant energy content. The mixed dung and straw is digested in the reactor. After digestion, remaining straw parts are separated in the separator and entered into the anaerobic tank for hydrolysis.

The hydrolysis of material after digestion in the first reactor increases the amount of produced gas by 20% to 80% compared to the amount of gas produced in the first reactor without a subsequent anaerobic hydrolysis process. Typically, the amount of gas produced according to the present invention is expected to increase by 25-50%.

Transportation of material by pumping using common biomass pumps requires that the dry matter content of the pumped material be kept below app. 15% dry matter. At a larger cost, worm conveyors may be provided for pumping material with a dry matter content of up to app. 25-30%. If the facility receives waste material with a high dry matter content, further waste material, such as straw, may not be added into the first reactor, but may instead be added to the content of the hydrolysis tank. Surprisingly, it has been found that feeding cut straws directly into the anaerobic hydrolysis tank results in a substantially homogenous mixture of straw and liquid in the tank, including a significantly reduced tendency for the straw to produce swim layer during downstream processing.

Depending on dry matter content, the output of the hydrolysis tank may be fed back into the first reactor, or, a separate second reactor for digestion of the hydrolysed material may be provided.

In an embodiment of the invention, gas produced in the hydrolysis tank is also provided to the first or second reactor or to the biogas handling and treatment system for further improvement of the biogas producing and treatment process.

During digestion of waste material in the reactor, various gases and compositions are produced, among these hydrogen sulphide and ammonia/ammonium.

Hydrogen sulphide originates from sulphate salts and proteins wherein amino acids may have some content of reduced sulphur. By digestion of biological substance, which takes place at neutral pH, the produced hydrogen sulphide will be present in the liquid where it is formed, and in the produced biogas.

Ammonia/ammonium is formed by digestion of urine and protein since urine has a high content of reduced nitrogen, and amino acids typically have a reduced N-group, the amino group.

In water at neutral pH, the ammonia and the hydrogen sulphide are partly soluble and react according to:
NH₃+H₂O=>NH₄⁺+OH⁻
H₂S+H₂O=>HS⁻+H⁺+H₂O(H₃O⁺)
The positive charge of NH₄⁺ and the negative charge of HS⁻ bring them together and:
NH₄⁺+HS⁻=>(NH₄⁺HS⁻)⁰

This salt is easily split into the corresponding gasses if the partial pressure of the gas over the liquid in which the salt is formed, is low for the two gasses. If the partial pressures of these gasses are high, the salt remains in the liquid.

During heating of biological substances in connection with the hydrolysis, a number of volatile compositions evaporate, such as organic acids, carbon dioxide, ammonia and hydrogen sulphide. These gasses are fed into the reactor or to the biogas handling and treatment system whereby the overall temperature in the biogas is increased. Hereby, it will be easier to maintain a constant and elevated pressure, since evaporated ammonia etc does not accumulate in the anaerobic tank including the tank headspace, but is output from the tank.

A pressure reduction caused by re-absorption of evaporated ammonia from the gasses in the liquid leads to formation of ammonium in accordance with the above-mentioned reactions.

Further, subsequent digestion of hydrolysed material may contain a significantly reduced content of ammonia/ammonium allowing the temperature at which the biogas production takes place to be higher.

In a livestock dung biogas producing facility, the gas produced typically has a high content of hydrogen sulphide, which it is required to reduce to avoid damaging of gas motors, etc, which transforms the biogas into electricity and heat. Since gas supplied from the hydrolysis tank has an increased temperature and contains evaporated water and ionised ammonium (NH₄⁺), the above-mentioned reaction takes place and converts the hydrogen sulphide to ammonium sulphide. Thus, the gas formed in the hydrolysis tank cleans the biogas produced in the reactor.

### BRIEF DESCRIPTION OF THE DRAWINGS

FIG. 1 schematically illustrates a biogas producing facility according to the present invention suited for waste having a low dry matter content,

FIG. 2 schematically illustrates a biogas producing facility according to the present invention suited for waste having a high dry matter content,

FIG. 3 schematically illustrates another biogas producing facility according to the present invention suited for waste having a high dry matter content, and

FIG. 4 schematically illustrates the hydrolysis tank of a biogas producing facility according to the present invention.

### DETAILED DESCRIPTION OF PREFERRED EMBODIMENTS

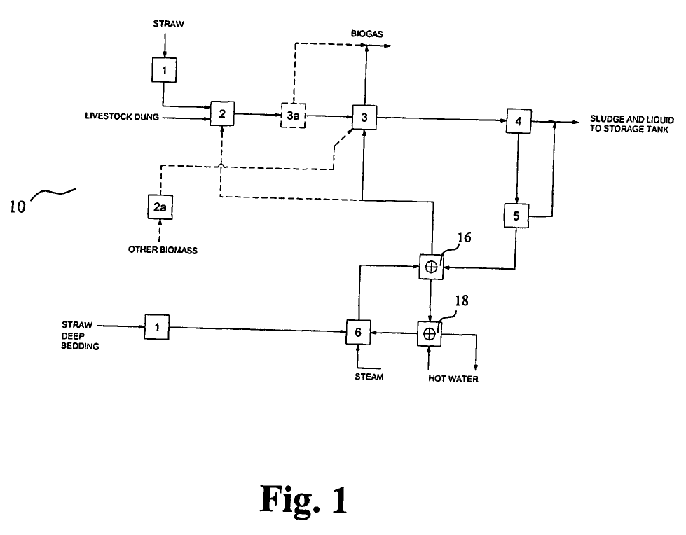

FIG. 1 schematically illustrates a biogas producing facility 10 for producing biogas from livestock dung mixed with organic waste, such as corn, grass, dry grass, fresh or dry straw, straw contained in livestock dung or in deep-bedding, silage, animal remains, etc. In the illustrated embodiment, the dung has low dry matter content so that a substantial amount of straw may be added to the dung. A partitioning device 1 cuts straw into straw parts having a mean length of approximately 5 to 10 cm. The cut straws and livestock dung are mixed in a tank 2, and the mixed matter is heat treated in a tank 3a, typically at 70-75° C., to kill unwanted bacteria. The heat-treated matter is fed into a first reactor 3 to be digested by bacteria for formation of biogas. Typically, the matter is digested for approximately 15-30 days depending on the reactor temperature. Typically, the reactor temperature ranges from 30° C.-55° C. A separator 4 separates particles larger than 0.2 cm to 2 cm, and the separated particles may be de-watered in a second separator 5 whereby the dry matter content reaches 10-15% dry matter. The separated matter is entered into the anaerobic hydrolysis tank 6 for anaerobic hydrolysis.

Optionally, the output from the separator 4 is entered into the anaerobic hydrolysis tank 6 through a heat exchanger 16. Then, the output from the hydrolysis tank constitutes the other medium of the heat exchanger 16 whereby the output from the hydrolysis tank is cooled before entrance into the first reactor 3.

Also optionally, the output from the separator 4 may be heated in a heat exchanger 18, e.g. by hot water, e.g. pressurized hot water, before entrance into the anaerobic hydrolysis tank 6.

Still optionally, organic waste, such as corn, grass, dry grass, fresh or dry straw, straw contained in livestock dung or in deep-bedding, silage, etc, may also be fed directly into the anaerobic hydrolysis tank 6, or, the organic waste may be mixed with at least some of the output from the first reactor 3 in a tank before entrance into the anaerobic hydrolysis tank 6.

For example, cut straw may be fed directly into the anaerobic hydrolysis tank 6. Surprisingly, it has been found this causes a substantially homogenous mixture of straw and liquid to be formed in the tank 6.

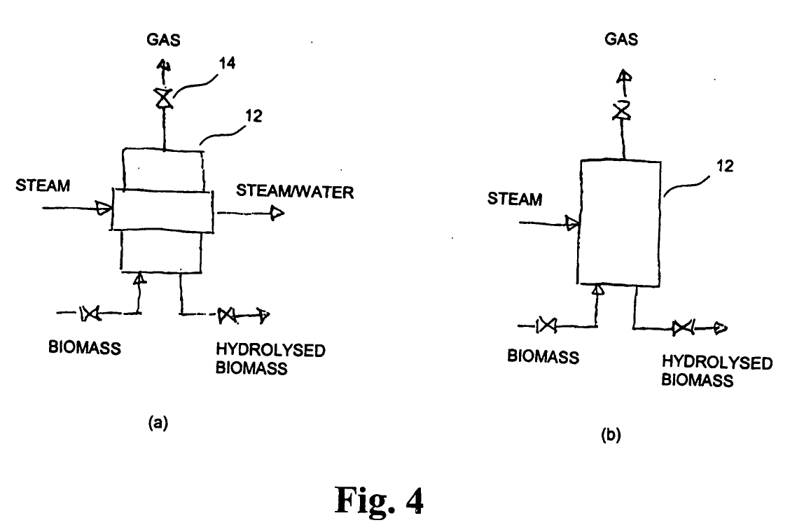

The anaerobic tank 6 may be pressurized by steam either directly or via a mantle as is further explained below with reference to FIG. 4, or, an increased pressure may be generated by the feeding pump feeding material into the anaerobic hydrolysis tank 6.

The hydrolysed matter is dissolved in liquid or takes the form of small particles.

Another biological substance 2a may be supplied to the facility 10, such as industrial waste, sorted household garbage, etc. This other biological substance is fed directly into the first reactor tank 3, and therefore it does not influence the other parts of the system.

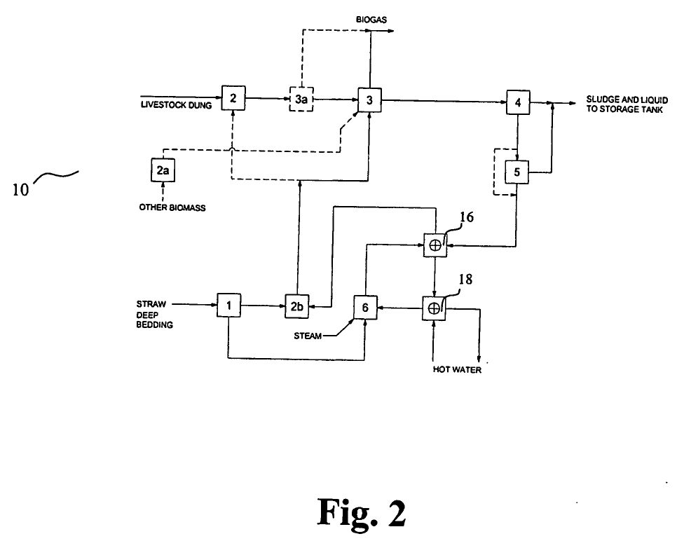

FIG. 2 schematically illustrates a biogas producing facility 10 for producing biogas from livestock dung mixed with straw. The mixed dung and straw has high dry matter content. A partitioning device 1 cuts straw into straw parts having a mean length of approximately 5 to 10 cm. The cut straws and hydrolysed material are mixed in a tank 2b, and the mixed matter is fed into a first reactor 3 to be digested by bacteria for formation of biogas. Alternatively or additionally, the cut straws may be entered directly into the anaerobic tank 6. Surprisingly, it has been found that a substantially homogenous mixture of straw and liquid is formed in the tank 6.

Livestock dung is mixed in 2 and heat-treated in 3a. The heat-treated matter is also fed into the first reactor 3 to be digested by bacteria for formation of biogas. Typically, the matter is digested for approximately 15-30 days depending on the reactor temperature. Typically, the reactor temperature ranges from 30° C.-55° C. A separator 4 separates particles larger than 0.2 cm to 2 cm and the separated particles may be de-watered in a second separator 5 whereby the dry matter content reaches 10-15% dry matter. The separated matter is entered into the hydrolysis tank 6 for hydrolysis.

Optionally, the output from the separator 4 is entered into the anaerobic hydrolysis tank 6 through a heat exchanger 16. Then, the output from the hydrolysis tank constitutes the other medium of the heat exchanger 16 whereby the output from the hydrolysis tank is cooled before entrance into the first reactor 3.

Also optionally, the output from the separator 4 may be heated in a heat exchanger 18, e.g. by hot water, e.g. pressurized hot water, before entrance into the anaerobic hydrolysis tank 6.

The anaerobic tank 6 may be pressurized by steam either directly or via a mantle as is further explained below with reference to FIG. 4, or, an increased pressure may be generated by the feeding pump feeding material into the anaerobic hydrolysis tank 6.

The hydrolysed matter is dissolved in the liquid or takes the form of small particles.

For livestock dung with a high content of dry mater, it may be unnecessary to de-water the separated particles. The dashed line indicates a bypass of the second separator 5.

Another biological substance 2a may be supplied to the facility 10, such as industrial waste, sorted household garbage, etc. This other biological substance is fed directly into the first reactor tank 3, and therefore it does not influence the other parts of the system.

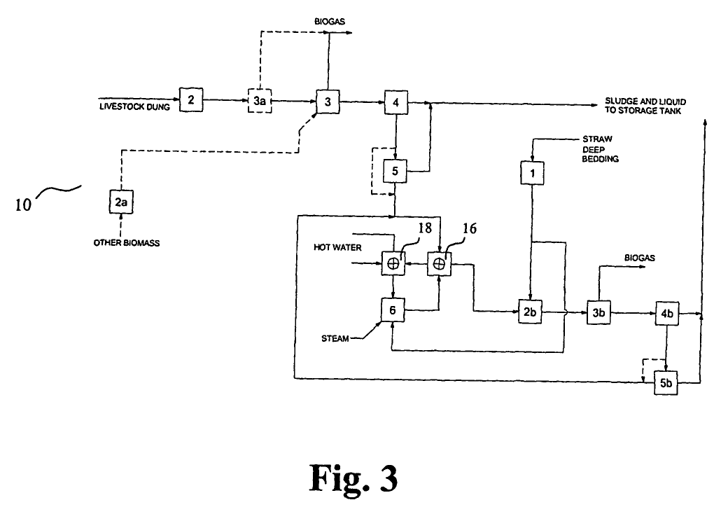

FIG. 3 schematically illustrates another biogas producing facility 10 for producing biogas from livestock dung mixed with straw. The mixed dung and straw has high dry matter content. Livestock dung is mixed in 2 and heat-treated in 3a at a temperature of about 70-75° C. The heat-treated matter is fed into a first reactor 3 to be digested by bacteria for formation of biogas. Typically, the matter is digested for approximately 15-30 days depending on the reactor temperature. Typically, the reactor temperature ranges from 30° C.-55° C. A separator 4 separates particles larger than 0.2 cm to 2 cm and the separated particles may be de-watered in a second separator 5 whereby the dry matter content reaches 10-15% dry matter. The separated matter is entered into the hydrolysis tank 6 for hydrolysis.

The anaerobic tank 6 may be pressurized by steam either directly or via a mantle as is further explained below with reference to FIG. 4, or, an increased pressure may be generated by the feeding pump feeding material into the anaerobic hydrolysis tank 6.

The hydrolysed matter is dissolved in the liquid or takes the form of small particles.

A partitioning device 1 cuts straw into straw parts having a mean length of approximately 5 to 10 cm. The cut straws and hydrolysed material from tank 6 are mixed in a tank 2b. The mixture is digested in a second reactor 3b. A separator 4b separates particles larger than 0.2 cm to 2 cm, and the separated particles may be de-watered in another separator 5b whereby the dry matter content reaches 10-15% dry matter. The separated matter is entered into the hydrolysis tank 6 for hydrolysis together with the output from the first reactor 3.

Alternatively or additionally, the cut straws may be entered directly into the anaerobic tank 6. Surprisingly, it has been found that a substantially homogenous mixture of straw and liquid is formed in the tank 6.

The hydrolysed matter is dissolved in the liquid or takes the form of small particles.

Optionally, the output from the separator 4 and the output from separator 4b are entered into the anaerobic hydrolysis tank 6 through a heat exchanger 16. Then, the output from the hydrolysis tank constitutes the other medium of the heat exchanger 16 whereby the output from the hydrolysis tank 6 is cooled before entrance into the first reactor 3.

Also optionally, the output from the separator 4 may be heated in a heat exchanger 18, e.g. by hot water, e.g. pressurized hot water, before entrance into the anaerobic hydrolysis tank 6.

For livestock dung with a high content of dry mater, it may be unnecessary to de-water the separated particles. A bypass of the second separator 5b is indicated by the dashed line.

Another biological substance 2a may be supplied to the facility 10, such as industrial waste, sorted household garbage, etc. This other biological substance is fed directly into the first reactor tank 3, and therefore does not influence the other parts of the system.

FIG. 4 schematically illustrates the hydrolysis tank of an embodiment of the invention wherein the gas formed during the hydrolysis is output to the reactor or the biogas handling and treatment system. Hereby, the biogas produced by the digestion is cleaned as explained above, and the temperature of the gas in the system is increased so that the efficiency of the biological cleaning process or a similar process may be increased.

In the illustrated embodiment, biological material to be hydrolysed is input to the hydrolysis tank 12. Depending on the desired hydrolysis temperature, the anaerobic tank is heated by steam injected directly into the tank as illustrated in FIG. 4b or by heating a mantle or pipes surrounding the tank as illustrated in FIG. 4a. Alternatively or additionally, the input entered into the anaerobic hydrolysis tank 12 through a heat exchanger (not shown). Then, the output from the hydrolysis tank constitutes the other medium of the heat exchanger whereby the output from the hydrolysis tank is cooled before entrance into the reactor. Also optionally, the input to the tank 12 may be further heated in a second heat exchanger (not shown), e.g. by hot water, e.g. pressurized hot water, before entrance into the anaerobic hydrolysis tank 12.

During temperature increase in the tank, the hydrolysis gas output valve 14 is open so that gas formed by the hydrolysis process in the headspace above the biological material communicates with gas formed by digestion in the reactor (not shown). When the biological liquid has reached the decided temperature, communication with the biogas produced in the reactor may be maintained at least for at predetermined period. If the pressure is to be increased, the valve 14 is closed, and when the desired pressure is reached, the valve and the supply of heat is controlled to maintain a substantially constant pressure in the tank. During hydrolysis under pressure, CO₂ and other gasses are formed by auto oxidation of organic material and dissolved in the liquid and in bacteria in the liquid. Upon pressure release, the pressure of dissolved gasses contained in the bacteria will disrupt the bacteria membranes and thus, destroy the bacteria.

Having finished hydrolysis, the headspace valve 14 may again be opened to avoid low pressure (vacuum) in the anaerobic tank. The temperature in the anaerobic tank may be decreased by release of steam to the reactor gas or the gas handling system, or, cooling may be effected utilising heat exchange or heat recovery.

Gas produced by the hydrolysis contains ammonia, hydrogen sulphide, carbon dioxide, Volatile Fatty Acids (VFA), evaporated water, etc. At the temperatures of the biogas in the headspace of the reactor and/or in the biogas handling and treatment system, these gasses condense and form ionised substances as explained above. The ionised substances react with each other and form salts. The gas is cooled and substantially saturated with evaporated water so that significant amounts of gasses that are not desired to be contained in the produced biogas will be absorbed in the condensed liquid.

In the embodiments illustrated in FIGS. 1-3, the separators 4, 4b separate particles larger than a threshold value that is set in accordance with the type of material digested in the reactor. For example, for hydrolysis of sludge from wastewater treatment plants, the threshold size is in the range from approximately 1.0 mm to approximately 2.0 mm, and for hydrolysis of fibre containing material, such as straw, the threshold size is in the range from approximately 0.2 cm to approximately 2.0 cm. The smaller particles have a high content of substances that cannot be microbially digested and a high content of salts of phosphor and nitrogen that desirably should not participate in the hydrolysis.

The separator may operate by sedimentation. However, sedimentation is not efficient in separating phosphor so lamella separators or vibrator screens etc may be preferred.

The output of the separator constitutes a liquid particle fraction of approximately 15-30 volume % of the separator input and contains approximately 20-50% of the dry matter of the separator input and has a dry matter content of approximately 8-15%.

If necessary, the second separators 5, 5b, increase the dry matter content to in the order of 10-15% depending on whether the biogas producing facility is intended for livestock dung with a low dry matter content, or for livestock dung with high dry matter content. The separator 5, 5b may be a centrifuge or a screw press, etc.

The output of the separator 5, 5b constitutes a liquid particle fraction of 60-70 volume % of the separator input and contains 70-80% of the dry matter of the separator input and has a dry matter content of 12-15%.

In the illustrated embodiments, the separation efficiency may be enhanced by adding precipitation agents or polymers, enhancing the particle size upstream the separation unit, and thus the retention of solids for downstream hydrolysis.

Laboratory experiments with wastewater treatment plant biological excess sludge show that biogas production using anaerobic digestion and subsequent anaerobic hydrolysis provides an enhancement of the biogas production by 50 to 70% compared to the production by similar anaerobic digestion without anaerobic hydrolysis. Similar experiments with animal manure or animal manure with added straw show that biogas production using anaerobic digestion and subsequent anaerobic hydrolysis provides an enhancement of the biogas production by 20 to 80% compared to the production by similar anaerobic digestion without anaerobic hydrolysis. Naturally, the dry matter reduction corresponds to the biogas production.

1. A biogas producing facility comprising a first reactor for holding organic waste for production of biogas by digestion and having an output for digested waste, and an anaerobic tank that is connected to the first reactor output for anaerobic hydrolysis of the digested waste and having an output for hydrolyzed material that is connected to an input of a second reactor for adding hydrolysed material to the content of the second reactor.

2. A biogas producing facility according to claim 1, wherein the anaerobic hydrolysis is: performed at a pressure that is substantially equal to the saturation vapour pressure during a period of the anaerobic hydrolysis.

3. A biogas producing facility comprising a first reactor for holding organic waste for production of biogas by digestion and having an output for digested waste, a separator that is connected to the first reactor output for selective separation of particles larger than a predetermined threshold size from the digested waste and having an output for the separated large particles, and an anaerobic tank that is connected to the separator output for anaerobic hydrolysis of the separated particles and having an output for hydrolysed material that is connected to an input of a second reactor for adding the hydrolysed material to the content of the I second reactor.

4. A biogas producing facility according to claim 1, wherein the first I reactor also constitutes the second reactor.

5. A facility according to claim 4, wherein the separation efficiency is enhanced by adding precipitation agents or polymers upstream the separator whereby the particle size upstream the separator is increased leading to improved retention of solids for downstream hydrolysis.

6. A facility according to claim 1, wherein the anaerobic tank further I comprises an input for reception of organic waste material in the tank for anaerobic hydrolysis together with digested material from the first reactor.

7. A facility according to claim 1, wherein the hydrolysis is performed! at a temperature in the range from 50° C.-75° C. for 0,25 to 24 hours.

8. A facility according to claim 1, wherein the hydrolysis is performed at a temperature in the range from 70° C.-100° C. for 0,25 to 16 hours.

9. A facility according to claim 1, wherein the hydrolysis is performed at a temperature in the range from/100° C.-125° C. for 0.25 to 8 hours.

10. A facility according to claim 1, wherein the hydrolysis is performed at a temperature in the range from 125° C.-150° C. for 0.25 to 6 hours.

11. A facility according to claim 1, wherein the hydrolysis is performed at a temperature in the range from 150° C.-175° C. for 0.25 to 4 hours. A facility according to claim 1 or 2, wherein the hydrolysis is performed at a temperature in the range from 175° C.-200° C. for 0.25 to 2 hours.

12. A facility according to claim 4, wherein the threshold size is larger than or equal to 0.1 cm.

13. A facility according to claim 4, wherein the threshold size is larger than or: equal to 0.2 cm.

14. A facility according to claim 4, wherein the threshold size is larger than or equal to 0.5 cm.

15. A facility according to claim 4, wherein the threshold size is larger than or equal to 1.0 cm.

16. A facility according to claim 4, wherein the threshold size is larger than or equal to 1.5 cm.

17. A facility according to claim 4, wherein the threshold size is larger than or! equal to 2.0 cm.

18. A facility according to claim 1, wherein the anaerobic tank is further connected to a pressure source for provision of a pressure in the anaerobic tank above 1 atmosphere.

19. A facility according to claim 4, wherein the separator further comprises a dewatering device for dewatering of the separated particles.

20. A facility according to claim 1, further comprising a partitioning I device for partitioning of organic waste and having an output for supplying the: partitioned waste to the reactor.

21. A facility according to claim 1, wherein a first waste material with high dry matter content is mixed with livestock dung and the mixture is entered into the! reactor for biogas production.

22. A facility according to claim 21, wherein the first waste material is straw.

23. A facility according to claim 1, wherein a first waste material with high dry matter content is mixed with hydrolysed material from the anaerobic tank and the mixture is input to the reactor.

24. A facility according to claim 23, wherein the first waste material is straw.

25. A facility according to claim 1, wherein a first waste material with high dry: matter content is mixed with hydrolysed material from the anaerobic tank and the mixture is input to the second reactor for digestion of the mixture.

26. A facility according to claim 25, further comprising a second separator that is connected to the second reactor output for selective separation of particles larger than: a predetermined threshold size from the digested waste and having an output for the separated large particles, and wherein the anaerobic tank is connected to the second separator output for hydrolysis of the separated particles.

27. A facility according to claim 26, wherein the second separator further comprises a second dewatering device for dewatering of the separated particles.

28. A facility according to claim 25, wherein the first waste material is straw.

29. A facility according to claim 1, wherein the anaerobic tank has a I gas output for supplying gas produced during hydrolysis to be combined with biogas produced in the reactor.

30. A method of producing biogas comprising the steps of producing biogas by digestion of organic waste in a reactor, subsequently performing an anaerobic hydrolysis of digested waste in an anaerobic hydrolysis tank, and feeding the hydrolysed material back into the reactor for further digestion and gas I production.

31. A method according to claim 30, further comprising the step of selective separation of particles larger than a predetermined threshold size from the digested waste before performing the anaerobic hydrolysis.
